(19) United States
(12) Patent Application Publication
(10) Pub. No.: US 2008/0250780 A1
(43) Pub. Date: Oct. 16, 2008

(54) Aquatic sink for carbon dioxide emissions with biomass fuel production

(76) Inventor: Howard A. Fromson, Stonington, CT (US)

Correspondence Address: ALIX YALE & RISTAS LLP, 750 MAIN STREET, SUITE 1400, HARTFORD, CT, 06103, US

(21) Appl. No.: 11/786,932

(22) Filed: Apr. 13, 2007

(51) Int. Cl. F23J 15/04; C12P 1/00

(52) U.S. Cl. 60/310; 110/215; 110/345; 435/41

(57) ABSTRACT

Carbon dioxide emissions from a hydrocarbon combustor are discharged into a large aquatic body, which acts as a CO₂ sink. The aquatic capture of the CO₂ prevents that CO₂ from entering the atmosphere. In addition, the captured CO₂ participates in a photosynthesis process for growing a plant bloom which can be harvested, and converted into a fuel for reuse in the combustion unit. The combustion in fossil fueled power plants yields two products: the thermal energy for power, and waste CO₂, which can be a raw material for growing an aquatic biomass. When the exhaust gases are discharged to the atmosphere, this raw material is lost, but by capturing this raw material in a highly efficient manner it can be converted to a usable form. An additional benefit of this efficient capture is that the adverse environmental effects of CO₂ emissions into the atmosphere are avoided.

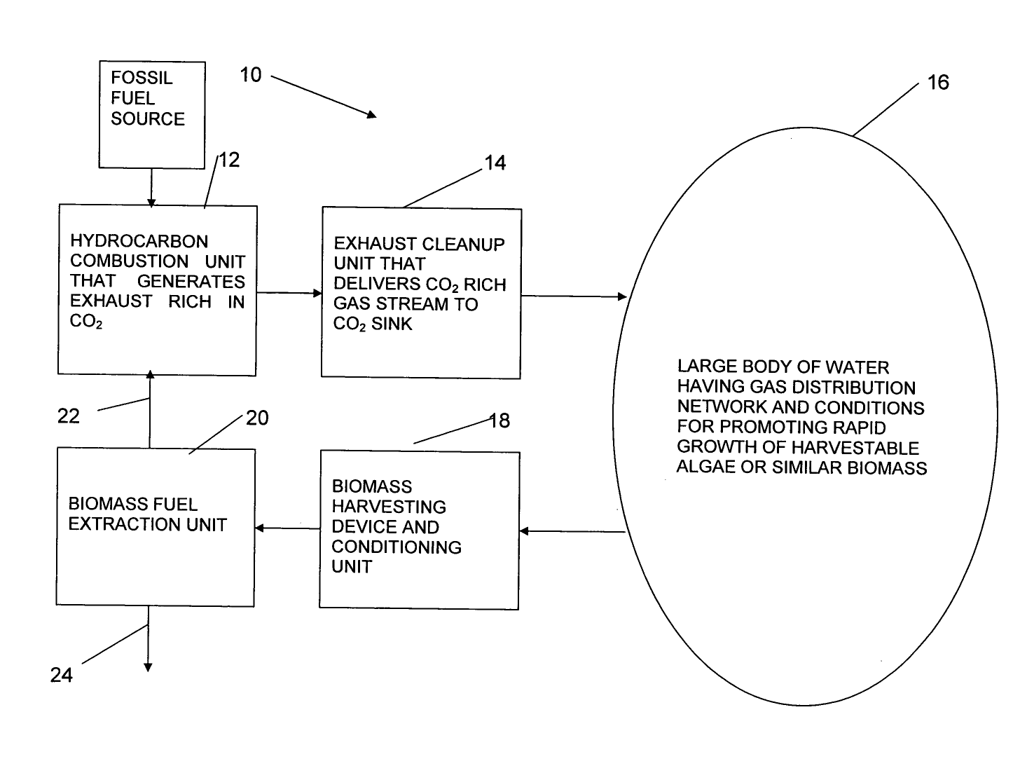

### BACKGROUND

The present invention relates to the treatment of carbon dioxide emissions from the combustion of hydrocarbons, and in particular, to the treatment of such emissions from large scale combustion units.

Scientists and government policy makers are expressing growing concern about the effects on the global environment, of the continuing increase in the release of man-made waste materials into the atmosphere. One source of such concerns, is the release of carbon dioxide (CO₂) as a byproduct of the combustion of hydrocarbon fuels. CO₂ is emitted in relatively low quantities by each of many individuals, such as by driving automobiles and burning fuel to heat homes. Larger emitters can be found in many industrials sites where fuels are burned to generate heat necessary for sustaining metallurgical and other chemical reactions. Emissions on a very large scale are produced by the burning of hydrocarbon fuel such as coal, oil, or natural gas in central electric generating stations, i.e., power plants.

Recent estimates of the annual production of CO₂ from the combustion of fossil fuels range as high as 1.7 billion tons. According to U.S. Pat. No. 3,999,329 the typical flu gas from a thermal power generating station utilizing coal, contains about 21% CO₂, 70% N₂, 5% water, and 2% oxygen along with significantly lower percentages of sulfur oxides and nitrous oxides.

In general, such CO₂ emissions have three natural sinks. The first is the upper levels of the atmosphere, the second is terrestrial plant life which through photosynthesis converts the CO₂ into carbohydrates, and the third is via absorption at the surface of the oceans, which converts the CO₂ into carbonic acid. Efforts at reducing CO₂ in the atmosphere have been largely focused on reducing energy demand, improving the efficiency of combustion processes, and reducing the CO₂ content of combustion exhaust before release into the atmosphere.

As discussed in U.S. Pat. No. 6,667,171 some have suggested the sequestration of CO₂ in large bodies of water, deep mines, or outdoor ponds, but have also recognized associated problems. U.S. Pat. No. 6,477,841 describes a method of converting solar energy stored via photosynthesis in macroalgae, into electrical energy.

### SUMMARY

The present invention takes a related but broadly different approach to the overall objective of reducing the level of CO₂ in the atmosphere.

According to the inventor, the combustion process in fossil fueled power plants can be viewed as yielding two products: the thermal energy that is the desired product for most power generating plants, and waste CO₂, which can be a raw material used in the process for growing an aquatic biomass. In the case where exhaust gases are discharged to the atmosphere, this raw material is lost. The invention is directed at capturing this raw material in a highly efficient manner and in a usable form. An additional benefit of this efficient capture is that the adverse environmental effects of CO₂ emissions into the atmosphere are avoided. The efficient capture of the CO₂ facilitates a more efficient growth of the biomass. The biomass is harvested and rendered usable as a fuel source or for other means.

It can thus be understood that in accordance with the present invention, a system and method are provided in which CO₂ emissions from a hydrocarbon combustion unit are discharged into a large body of water, which acts as a CO₂ sink. The capture of the CO₂ in the water prevents that CO₂ from entering the atmosphere. In addition, the CO₂ in the water participates in a photosynthesis process for growing a plant bloom in the water which can be harvested, and converted into a fuel for reuse in the combustion unit, or elsewhere.

Accordingly, in one embodiment, the invention is directed to a system for processing CO₂ emissions, comprising a hydrocarbon combustion unit that generates an exhaust stream containing CO₂ gas. A gas distribution system connects the combustion unit with a large body of water, for discharging a plume of the gas into the water. A plant bloom grows in the CO₂ plume in the body of water. A plant bloom harvesting system removes a portion of the bloom and accumulates a biomass outside the body of water. A biomass fuel extraction unit converts the biomass into a hydrocarbon fuel.

A method embodiment comprises the steps of combusting hydrocarbons to generate an exhaust stream containing CO₂ gas, and distributing the gas as a plume in a large body of water. A plant bloom is grown in the plume, and is harvested to produce a biomass that is converted into a hydrocarbon fuel. Preferably, the combustion unit is a stationary electric power generation station, and the biomass conversion unit for producing the biofuel is located at the power generating station.

In the preferred embodiment, the recovered biomass will most typically supplement the conventional fossil fuel source. There is no process that is 100% efficient in capturing and utilizing the CO₂ to produce the biomass, and some of the thermal energy produced must be invested to render the biomass useful as a fuel source. The present invention would use the recovered biomass to offset some of the fossil fuel requirements.

Although the invention is especially suitable as a system and method to be implemented at or near the shore of a large body of salt water such as an ocean, sea, bay or cove, implementation is also possible inland, even in desert areas, or other areas where the sun shines steadily and thereby maximizes the photosynthesis process that drives the growth and carbohydrate content of the plants that will be converted into biofuel. Moreover, the process may be sufficiently cost effective to justify creation of a dedicated body of water alongside the power station, in an otherwise arid location.

### BRIEF DESCRIPTION OF THE DRAWING

The accompanying FIGURE is a schematic representation of the system and process according to the preferred embodiment of the present invention.

### DETAILED DESCRIPTION

The FIGURE shows a system 10 having a hydrocarbon combustion unit 12 that generates exhaust rich in CO₂. The combustion unit is preferably a stationary central power generating station, that burns coal, natural gas, or oil, of any burner type including fluidize bed, but can also include other stationary units located in factories, waste reprocessing plants, or institutional facilities including cogeneration plants. Such units typically have an exhaust cleanup unit 14 for reducing the particulates, nitrate oxides (NOx) and possibly CO₂, but presently, almost all such exhaust as emitted from, e.g., a stack or chimney, contains substantial quantities of CO₂.

With the exhaust gas preferably cleaned of particulates and other potential containments, the exhaust gas is pumped or otherwise delivered to a gas distribution system that leads to a large body of water having a distribution network and conditions for promoting rapid growth of harvestable algae or similar biomass.

U.S. Pat. No. 5,309,672, “Submerged Platform Structure for Open Ocean Macroalgal Farm Systems”, describes an open ocean farm structure for attachment of macroalgal plants. The frame structure is made up of linear elements connected with nodes to form a three dimensional truss. The linear elements are composed of tubes containing solid rods which are screw connected to the nodes. The ends of the tubes abut the nodes so that screwing the rods into the nodes puts the tubes in compression. The truss structure thus formed is strong and flexible. Because the truss structure is made of tubes having minimal cross sectional area, the structure is relatively transparent to the forces of wave motion. The disclosure of this patent is hereby incorporated by reference, and is merely representative of the enhancements that can be provided in the body of water for promoting the rapid growth of harvestable algae.

The piping and nozzles for discharging the CO₂-laden exhaust gas in an ideal pattern and volume to produce a plume optimized for use in conjunction with, for example, the submerged platform structure described above, would be well within the ordinary skill of engineers and craftsman who design and assemble gas handling and distribution systems, and marine biologists taking into account the depth, salinity, temperature range, wave motion, and type or types of algae or similar blooming plant material to be grown. Other factors are the latitude and seasonal changes and thus variations of the intensity and penetration of sunlight as well as the prevalence of sunlight relative to cloudy or other less desirable conditions for photosynthesis process by which the plants produce carbohydrates using the sunlight and CO₂.

In some circumstances, an artificial body of water can be created to provide the large sink for the CO₂ content of the discharged exhaust.

It should be appreciated that an important purpose of the main body of water is to serve as a sink for absorbing essentially all of the CO₂ in the discharged exhaust stream. For a large body of water such an ocean, bay, or the like, it is not necessary that a balance be maintained between the rate of CO₂ deposited in the body, and the rate of CO₂ utilized in the photosynthesis process. However, if a dedicated body of water associated with an inland combustion unit is employed in the system, the rate of discharge into the body of water should more closely match the rate of utilization of CO₂ in the biomass.

It should thus be appreciated that, ideally, all of the CO₂ rich gas stream from the combustion unit is discharged into the body of water which acts as a CO₂ sink, preventing the CO₂ from entering the atmosphere, and that all or most of that CO₂ in the body of water participates in the photosynthesis process, thereby preventing excess build up of CO₂ in the body of water.

The algae bloom can be harvested using known techniques. The harvesting devise will of course have an active front end which removes the algae from the bloom or from the stationary position if grown on a latticework, and a back end on land where conditioning, such as washing and/or drying and other forms of cleaning can be performed. Such drying can be implemented using some of the exhaust stream from the exhaust cleanup unit 14 or the combustion unit 12, in a system where the biomass processing is in the same station as the combustion unit 12.

The conditioned biomass is transferred to the biomass fuel extraction unit 20, where the carbohydrates are converted into a usable fuel and preferably delivered back to the combustion unit via line 22, or to the extent of any excess, packaged for offsite use via line 24. U.S. Pat. No. 4,341,038 describes a method for obtaining oil products from algae. In particular, oil products and a high nitrogen content residue are obtained by growing halophilic algae in saline solution, harvesting an algae-saltwater slurry, solvent extracting the slurry, then recovering the product and residue. According to this patent and with further reference to U.S. Pat. No. 4,115,949, such algae can be cultivated in order to obtain hydrocarbon mixtures essentially similar to fossil oil. The disclosures of these patents are hereby incorporated by reference.

1. A system for processing carbon dioxide emissions comprising:
a hydrocarbon combustion unit that generates an exhaust stream containing carbon dioxide gas;
a gas distribution system connecting the combustion unit with a large body of water, for discharging a plume of the gas into the water;
a plant bloom growing in the plume in the body of water;
a plant bloom harvesting system that removes a portion of the bloom and accumulates a biomass outside the body of water; and
a biomass fuel extraction unit that converts the biomass into a hydrocarbon fuel.

2. The system of claim 1, wherein the gas distribution system includes means for removing contaminants other than carbon dioxide from the exhaust stream.

3. The system of claim 1, wherein the bloom harvesting system includes means for conditioning the biomass before delivery to the fuel extraction unit.

4. The system of claim 1, wherein at least some of the biomass converted into fuel is continually delivered to the hydrocarbon combustion unit.

5. The system of claim 1, wherein the body of water is a saltwater ocean, sea, bay, or cove.

6. The system of claim 1, wherein
the combustion unit is a stationary electric power generating station; and
the fuel extraction unit is located at the power generating station.

7. The system of claim 4, including
a fossil fuel source;
means for feeding the fossil fuel to the combustion unit;
whereby the combustion unit simultaneously combusts said fossil fuel and said biomass fuel.

8. The system of claim 1, wherein
the combustion unit is a stationary fossil fuel electric power generating station;
the fuel extraction unit is located at the power generating station;
at least some of the biomass converted into fuel is continually delivered to the hydrocarbon combustion unit and combusted simultaneously with the fossil fuel.

9. The system of claim 8, wherein the body of water is a saltwater ocean, sea, bay, or cove.

10. The system of claim 1 wherein
the gas distribution system includes means for removing contaminants other than carbon dioxide from the exhaust stream;
the body of water is a saltwater ocean, sea, bay, or cove;
the bloom harvesting system includes means for conditioning the biomass before delivery to the fuel extraction unit; and
at least some of the biomass converted into fuel is continually delivered to the hydrocarbon combustion unit.

11. The system of claim 8, wherein
the gas distribution system includes means for removing contaminants other than carbon dioxide from the exhaust stream;
the body of water is a saltwater ocean, sea, bay, or cove;
the bloom harvesting system includes means for conditioning the biomass before delivery to the fuel extraction unit; and
all the biomass converted into fuel is continually delivered to the hydrocarbon combustion unit.

12. The system of claim 8, wherein the biomass fuel extraction unit converts the biomass into a hydrocarbon fuel oil.

13. The system of claim 10, wherein the biomass fuel extraction unit converts the biomass into a hydrocarbon fuel oil.

14. A method for processing carbon dioxide emissions comprising:
combusting hydrocarbons to generate an exhaust stream containing carbon dioxide gas;
distributing the gas as a plume in a large body of water;
growing a plant bloom in the plume;
harvesting a portion of the bloom and accumulating a harvested biomass outside the body of water; and
converting the harvested biomass into a hydrocarbon fuel.

15. The method of claim 14, wherein the biomass is converted into a hydrocarbon fuel oil and is continually delivered to the hydrocarbon combustion unit.

16. The method of claim 14, wherein the body of water is a saltwater ocean, sea, bay, or cove.

17. A method for operating a fossil fueled central power generating station, comprising:
combusting fossil fuel to generate heat for producing power and an exhaust stream containing carbon dioxide gas;
distributing the gas as a plume in a large body of water;
growing a plant bloom in the plume;
harvesting a portion of the bloom and accumulating a harvested biomass outside the body of water;
converting the harvested biomass into a hydrocarbon fuel; and
combusting converted hydrocarbon fuel with the fossil fuel to generate additional heat for producing power and additional carbon dioxide in said exhaust stream.

18. The method of claim 17, wherein the body of water is a saltwater ocean, sea, bay, or cove.

19. The method of claim 17, wherein all the biomass converted into hydrocarbon fuel is combusted with the fossil fuel.

20. The method of claim 18, wherein all the biomass converted into hydrocarbon fuel is combusted with the fossil fuel.
